(19) United States
(12) Patent Application Publication
(10) Pub. No.: US 2021/0353225 A1
(43) Pub. Date: Nov. 18, 2021

(54) SYSTEMS AND METHODS FOR DETECTING ORAL CANCER USING MOLECULAR CHEMICAL IMAGING

(71) Applicant: CHEMIMAGE CORPORATION, Pittsburgh, PA (US)

(72) Inventors: Shona STEWART, Pittsburgh, PA (US); Aaron SMITH, Monroeville, PA (US); Patrick J. TREADO, Pittsburgh, PA (US)

(21) Appl. No.: 17/323,462

(22) Filed: May 18, 2021

Related U.S. Application Data
(60) Provisional application No. 63/026,447, filed on May 18, 2020.

(51) Int. Cl. A61B 5/00; A61B 1/07; A61B 1/06; A61B 1/05; A61B 1/04; A61B 1/24

(52) U.S. Cl. CPC A61B 5/4887; A61B 1/07; A61B 1/063; A61B 1/24; A61B 1/0646; A61B 1/051; A61B 1/043; A61B 1/0684

(57) ABSTRACT

Methods and systems of identifying oral cancer in vivo are disclosed. An oral cavity of a patient is illuminated with a plurality of illuminating photons. A plurality of interacted photos are received from the oral cavity. The interacted photons may have been absorbed, reflected, scattered or emitted by the oral cavity. The interacted photons are filtered into first and second polarized multi-passband wavelengths using first and second tunable conformal filters, respectively. A detector captures the first and second polarized multi-passband wavelengths. A processor automatically discriminates between cancerous tissue and non-cancerous tissue in an image resolved from the first and second polarized multi-passband wavelengths.

### CROSS-REFERENCE TO RELATED APPLICATIONS

The present application claims priority to U.S. Provisional Patent Application No. 63/026,447, titled SYSTEMS AND METHODS FOR DETECTING ORAL CANCER USING MOLECULAR CHEMICAL IMAGING, filed May 18, 2020, which is hereby incorporated by reference herein in its entirety.

### TECHNICAL FIELD

The present disclosure relates to systems and methods for detecting oral cancer using molecular chemical imaging. More particularly, the document discloses systems, devices and methods for detecting oral cancer using visible-near infrared or shortwave infrared reflectance molecular chemical imaging in the form of a handheld probe or endoscopic device.

### BACKGROUND

Oral cancer is a devastating disease. In the US, more than 50,000 new cases of cancer of the oral cavity and pharynx are diagnosed and more than 10,000 patients die from the disease each year. The optimal width of the surgical margin for oral cancer continues to be a matter of debate. Post-surgical histological analysis often indicates a need for resection as patients relapse despite having margins diagnosed as histologically negative. Researchers have demonstrated that complete resection of early stage (T1/T2N0) tongue cancer can lead to excellent oncologic outcomes. However, these results are lost if the margin of resection is positive at the time of initial surgery. The ability to clear margins with re-resection does not confer improved survival.

Other types of oral cancer have similar declines in survival rate and increase in costs for re-resection procedures. For example, the five-year survival rate for patients with oral squamous cell carcinoma (OSCC) without recurrence was 92%. In contrast, the survival rate with recurrent was merely 30%. In patients with head and neck cancer, six-month incremental adjusted total costs were 60 k per patient for metastatic head and neck cancers (mHNC) and 21 k per patient for recurring head and neck cancers (rHNC).

Intraoperative palpation by the surgeon is the current standard for assessment of surgical margins, along with frozen sections obtained intra-operatively. Unfortunately, tactile assessment of margin status is not sufficient to define the borders of a tumor intraoperatively, and frozen sections are time consuming Even though it is assumed that these positive margins can be cleared with re-resection, the data above suggests that re-resection may be associated with worse outcomes. The consequences of tumor-positive resection margins are significant because this often leads to revision surgery, the need for adjuvant therapy (post-operative radiotherapy), and higher morbidity and mortality.

Postoperative tumor recurrence leads to a poor prognosis and a poor quality of life, and therefore, successful development of a tool that can help avoid recurrence by successfully identifying tumor margins and remaining tumor(s) following the initial resection can improve surgical decision-making and patient outcomes.

Even though patients may be diagnosed as being “tumor margin negative” by histological confirmation, patients may still relapse. As such, a need exists for a non-invasive, contrast-free intraoperative surgical tool to detect tumor margin status in real time in order to improve outcomes for such patients.

### SUMMARY

The present disclosure is related to systems, devices and methods to develop real-time, intraoperative quantitative assessment of tumor margin status, which could improve treatment outcome. Molecular-specific, diagnostic optical imaging based on Visible Near Infrared (Vis-NIR, approx. 400-1100 nm) or short wave infrared imaging (SWIR, 1000-2000 nm) spectroscopy, applied as a high information content and real-time intraoperative imaging modality, can address a critical unmet need. The methods and systems described herein using molecular chemical imaging can provide adequate sensitivity and specificity to visualize tumors in vivo.

In one embodiment, there is a system for detecting oral cancer in vivo, the system comprising: an illumination source configured to generate illuminating photons; an imaging device comprising: a fiber optic bundle comprising a plurality of optical fibers configured to receive the illuminating photons from the illumination source and direct the illuminating photons to an oral cavity of a patient, and a lens configured to collect interacted photons from the oral cavity of the patient; a polarizing beam splitter configured to receive the interacted photons from the lens and split the interacted photons into at least a first plurality of interacted photons and a second plurality of interacted photons; a first tunable conformal filter configured to receive the first plurality of interacted photons and to generate first polarized multi-passband wavelengths; a second tunable conformal filter configured to receive the second plurality of interacted photons and to generate second polarized multi-passband wavelengths; a beam combiner configured to receive and combine the first and second polarized multi-passband wavelengths; a detector configured to receive the combined first and second polarized multi-passband wavelengths; and a controller configured to tune the first and second tunable conformal filters such that the first polarized multi-passband wavelengths and the second polarized multi-passband wavelengths discriminate between cancerous tissue and non-cancerous tissue in the oral cavity.

In another embodiment, the illumination source comprises at least one of a quartz tungsten halogen light, a metal halide light, a light emitting diode (LED), a LED array, a pulsed LED, a pulsed LED array, a laser, a pulsed laser, or a broadband illumination source.

In another embodiment, the system further comprises a first mirror configured to direct the first polarized multi-passband wavelengths from the first tunable conformal filter to the beam combiner; and a second mirror configured to direct the second polarized multi-passband wavelengths from the second tunable conformal filter to the beam combiner.

In another embodiment, the detector comprises at least one of a charge coupled device (CCD) detector, a complementary metal-oxide-semiconductor (CMOS) detector, an indium gallium arsenide (InGaAs) detector, a platinum silicide (PtSi) detector, an indium antimonide (InSb) detector, or a mercury cadmium telluride (HgCdTe) detector.

In another embodiment, the first polarized multi-passband wavelengths correspond to a background and the second polarized multi-passband wavelengths correspond to the cancerous tissue.

In another embodiment, the imaging device comprises an endoscope or a handheld probe.

In one embodiment, there is a method of detecting oral cancer in vivo, the method comprising: illuminating an oral cavity of a patient with a plurality of illuminating photons; receiving a plurality of interacted photons from the oral cavity of the patient; filtering the plurality of interacted photons into first polarized multi-passband wavelengths and second polarized multi-passband wavelengths using first and second tunable conformal filters, respectively; capturing, via a detector, the first and second polarized multi-passband wavelengths; and automatically discriminating between cancerous tissue and non-cancerous tissue in an image resolved from the first and second polarized multi-passband wavelengths.

In another embodiment, illuminating the oral cavity comprises illuminating the oral cavity with the plurality of illuminating photons from at least one of a quartz tungsten halogen light, a metal halide light, a light emitting diode (LED), a LED array, a pulsed LED, a pulsed LED array, a laser, a pulsed laser, or a broadband illumination source.

In another embodiment, the method further comprises: directing, via a first mirror, the first polarized multi-passband wavelengths from the first tunable conformal filter to a beam combiner; and directing, via a second mirror, the second polarized multi-passband wavelengths from the second tunable conformal filter to the beam combiner.

In another embodiment, the detector comprises at least one of a charge coupled device (CCD) detector, a complementary metal-oxide-semiconductor (CMOS) detector, an indium gallium arsenide (InGaAs) detector, a platinum silicide (PtSi) detector, an indium antimonide (InSb) detector, or a mercury cadmium telluride (HgCdTe) detector.

In another embodiment, the first polarized multi-passband wavelengths correspond to a background and the second polarized multi-passband wavelengths correspond to the cancerous tissue.

In another embodiment, oral cavity of the patient is illuminated with the plurality of illuminating photons via an endoscope or a handheld probe.

In one embodiment, there is an imaging system for detecting oral cancer in vivo, the imaging system for use with an illumination source and an imaging device, the illumination source configured to generate illuminating photons, and the imaging device configured to direct the illuminating photons to an oral cavity of a patient and collect interacted photons from the oral cavity of the patient, the system comprising: a polarizing beam splitter configured to receive the interacted photons from the imaging device and split the interacted photons into at least a first plurality of interacted photons and a second plurality of interacted photons; a first tunable conformal filter configured to receive the first plurality of interacted photons and to generate first polarized multi-passband wavelengths; a second tunable conformal filter configured to receive the second plurality of interacted photons and to generate second polarized multi-passband wavelengths; a beam combiner configured to receive and combine the first and second polarized multi-passband wavelengths; a detector configured to receive the combined first and second polarized multi-passband wavelengths; and a controller configured to tune the first and second tunable conformal filters such that the first polarized multi-passband wavelengths and the second polarized multi-passband wavelengths discriminate between cancerous tissue and non-cancerous tissue in the oral cavity.

In another embodiment, the illumination source comprises at least one of a quartz tungsten halogen light, a metal halide light, a light emitting diode (LED), a LED array, a pulsed LED, a pulsed LED array, a laser, a pulsed laser, or a broadband illumination source.

In another embodiment, the system further comprises: a first mirror configured to direct the first polarized multi-passband wavelengths from the first tunable conformal filter to the beam combiner coupled to the detector; and a second mirror configured to direct the second polarized multi-passband wavelengths from the second tunable conformal filter to the beam combiner.

In another embodiment, the detector comprises at least one of a charge coupled device (CCD) detector, a complementary metal-oxide-semiconductor (CMOS) detector, an indium gallium arsenide (InGaAs) detector, a platinum silicide (PtSi) detector, an indium antimonide (InSb) detector, or a mercury cadmium telluride (HgCdTe) detector.

In another embodiment, the first polarized multi-passband wavelengths correspond to a background and the second polarized multi-passband wavelengths correspond to the cancerous tissue.

In another embodiment, the imaging device comprises an endoscope or a handheld probe.

### BRIEF DESCRIPTION OF THE DRAWINGS

FIG. 1 illustrates an endoscope or handheld probe comprising an imaging system having a plurality of conformal filters in a dual polarization configuration in accordance with an embodiment;

FIG. 1A is an end-on view of the endoscope/probe according to the embodiment of FIG. 1;

FIG. 1B illustrates a patterned conformal filter configuration with a CCD detector in accordance with an embodiment;

FIG. 2 illustrates an endoscope/probe comprising an imaging system having a plurality of multivariate optical element (MOE) filters in accordance with an embodiment;

FIG. 2A is an end-on view of the endoscope/probe according to the embodiment of FIG. 2;

FIG. 2B is a cross-sectional view of the distal end of the endoscope/probe according to the embodiment of FIG. 2;

FIG. 3 illustrates an endoscope/probe comprising an imaging system having a conformal filter in accordance with an embodiment;

FIG. 3A is an end-on view of the endoscope/probe according to the embodiment of FIG. 3;

FIG. 4 illustrates an endoscope/probe comprising an imaging system having a plurality of conformal filters in a dual polarization configuration for source illumination modulation in accordance with an embodiment;

FIG. 4A is an end-on view of the endoscope/probe according to the embodiment of FIG. 4;

FIG. 4B is an end-on view of an alternate embodiment of the endoscope/probe according to the embodiment of FIG. 4;

FIG. 5 illustrates an endoscope/probe comprising an imaging system having an acousto-optic filter in accordance with an embodiment;

FIG. 5A is an end-on view of the endoscope/probe according to the embodiment of FIG. 5;

FIG. 6 illustrates an endoscope/probe comprising an imaging system having a MOE filter wheel in accordance with an embodiment;

FIG. 6A is an end-on view of the endoscope/probe according to the embodiment of FIG. 6; and

FIG. 7 illustrates an endoscope/probe comprising an imaging system having a patterned etalon filter arrangements in accordance with an embodiment.

### DETAILED DESCRIPTION

The present disclosure features intraoperative medical imaging systems which can assist surgeons in detecting oral cancer. In some embodiments, the intraoperative medical imaging systems can assist surgeons in detecting oral cancer in vivo, such as during a surgical procedure. The systems disclosed herein are suitable for use as stand-alone devices, or may be incorporated into other medical imaging devices such as a robotic platform. In one embodiment, the systems disclosed herein may be used in conjunction with an endoscope or a handheld probe. The medical imaging systems disclosed herein may provide real-time detection of tumors and anatomic structures during oral cancer surgical procedures. Generally, the systems disclosed herein provide for illuminating a tumor site within a patient's mouth, collecting photons that have interacted with the sample, detecting the interacted photons to generate an image data set of the sample, and analyzing the image data set. Interacted photons may comprise one or more of photons absorbed by a sample, photons reflected by a sample, photons scattered by a sample, and photons emitted by a sample. In one embodiment, the medical imaging system provides multivariate imaging. Multivariate imaging generates a plurality of wavelengths corresponding to a first image data set (T1) and a second image data set (T2). These first and second image data sets may be analyzed using an optical computation. Multivariate imaging creates enhanced image contrast and increased discrimination between a target and background. In certain embodiments, the first image data set and the second image data set feature hyperspectral image data. In some embodiments, the medical imaging systems feature imaging frame rates of >10 Hz (hyper-cubes/second).

Molecular chemical imaging provides many advantages over conventional methods of identifying surgical margins for oral cancer. For example, molecular chemical imaging is non-invasive, is able to penetrate tissue in vivo, provides a quantitative analysis of the surgical margin, is reagentless (i.e., does not require a contrast-enhancing agent), and provides real-time detection for cancerous tissue or tumors. Moreover, molecular chemical imaging may be adapted to surgical robotics settings, which may become more prevalent in the future.

The systems and methods disclosed herein may be used on various oral biological structures of a patient, such as the patient's tongue, gums, and/or palate, and/or other anatomical structures, physiological systems, cells, blood, fat, nerves, muscle, and the like. The systems disclosed herein may also be used on other portions of a patient's head and neck area whether internal and external.

Further, the systems and methods may be used to discriminate between two or more different biological samples. In one embodiment, the systems disclosed herein may be employed to differentiate cancer from normal tissue, determine one or more of a type of cancer, a cancer stage, a cancer progression and a cancer grade. In another embodiment, the systems and methods may be employed during surgical procedures to assist in the removal of remove cancerous tissue or tumors.

As disclosed herein, the systems of the present disclosure provide illumination to a biological tissue. It is known that such illumination may penetrate a biological sample up to several centimeters depending on wavelength and tissue type. Thus, such penetrating illumination permits the imaging of bodily fluids contained inside an anatomical structure. Further, the bodily fluids may be directly imaged where their presence resides outside of the anatomical structure or other biological sample.

The medical imaging instruments disclosed herein provide real-time multivariate imaging by generating a multivariate signal using one or more detectors. The detectors detect the multivariate signal to produce one or more image data sets. Provided herein are two ways to achieve this result. One such method includes illuminating a sample, collecting interacted photons that have interacted with the sample, and modulating the collected signal prior to passing the signal on to a detector. A second method includes modulating the illumination source signal prior to interaction with a sample, collecting interacted photons of the modulated signal, and detecting the interacted photons of the signal. Both processes provide a modulated signal to produce a multivariate chemical image in real-time with enhanced contrast to assist surgeons with delicate medical procedures. The embodiments contained herein can further be configured to provide real-time images displayed in stereo vision. Such a configuration would be apparent to those of skill in the art in view of this disclosure. Stereo vision further assists a surgeon by providing the depth perception needed in medical procedures employing medical imaging techniques, such as in endoscopic procedures. Systems and methods recited herein provide exemplary embodiments of the instant disclosure and are not intended to limit the disclosure to any particular embodiment.

In the following illustrated embodiments, like reference characters refer to like parts.

### Modulating Collected Optical Signal

The following embodiment features systems and methods for modulating an optical signal after the collection of photons that have interacted with a target oral cavity.

System Having Conformal Filters in a Dual Polarization Arrangement:

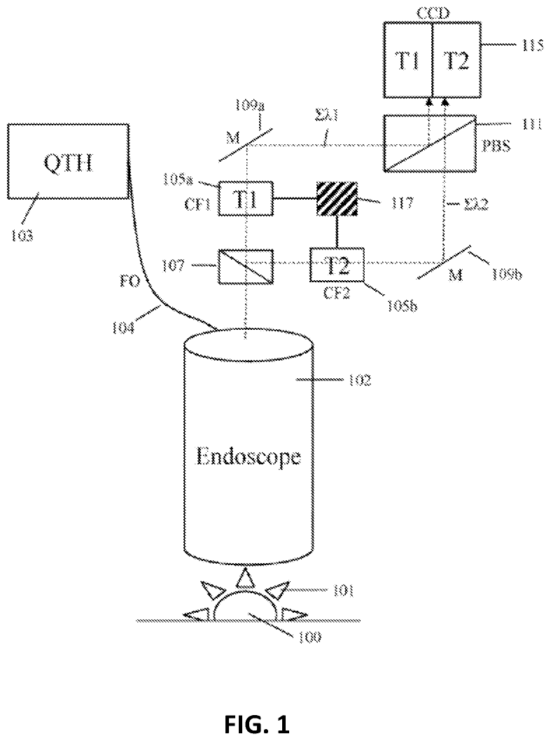

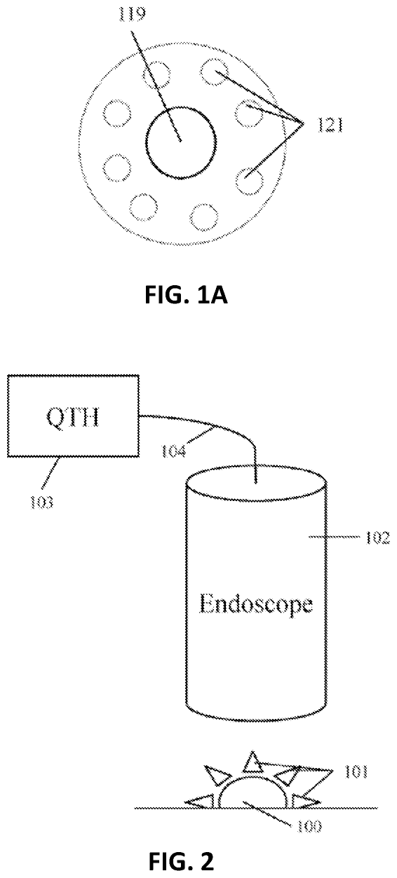

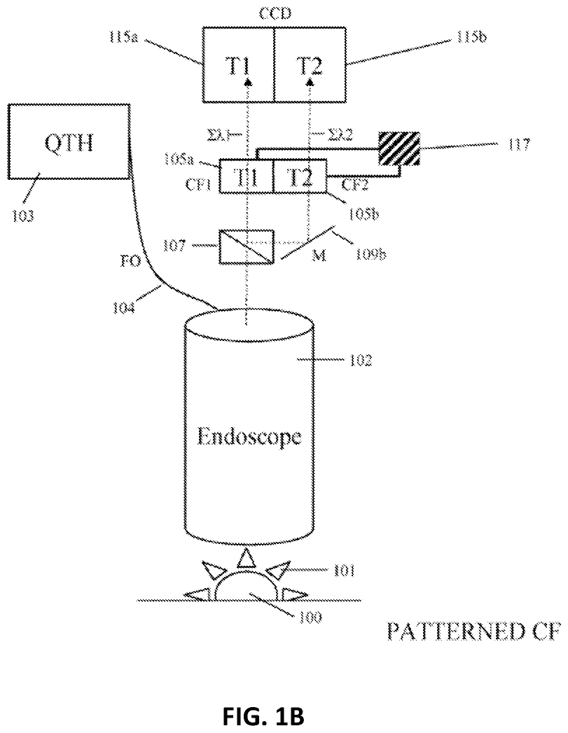

Referring now to FIG. 1, an oral cavity 100 of a patient may be illuminated and/or excited by an illumination source 103. In one embodiment, the illumination source 103 may comprise a quartz tungsten halogen light source. In other embodiments, the illumination source 103 may comprise a metal halide light source, a light emitting diode (LED), a LED array having a uniform selection of emitters which emit over a constant wavelength range or a plurality of emitters which emit over a diversity of wavelength ranges, a pulsed LED, a pulsed LED array, a laser, a pulsed laser, a broadband illumination source and/or the like. The illumination source 103 generates illuminating photons that are directed from the illumination source 103 to the distal end of an endoscope or handheld probe 102 (i.e., an imaging device) through a fiber optic bundle 104. The endoscope/probe 102 is configured to direct interacted photons 101 that have interacted with the oral cavity 100 to a polarizing beam splitter 107. First and second independently tunable conformal filters 105a, 105b are situated along distinct orthogonal beam paths to filter orthogonal polarization components emerging from the polarizing beam splitter 107. Suitable conformal filters may include those disclosed in U.S. Pat. No. 9,041,932 to Priore et al., filed Jan. 4, 2013, issued May 26, 2015, assigned to ChemImage Technologies LLC, and entitled CONFORMAL FILTER AND METHOD OF USE THEREOF, the entirety of which is hereby incorporated by reference.

In the depicted embodiment, the paths of the filtered beams are not parallel through the first and second conformal filters 105a, 105b, but are directed by appropriate reflectors, such as first and second mirrors 109a, 109b, to a beam combiner 111. In alternate embodiments, the beam combiner 111 may be a polarizing cube or polarizing beam splitter. In another embodiment, the orthogonal components may comprise the same or different multi-passband wavelengths (Σλ₁ and Σλ₂). In the exemplary embodiment, the first conformal filter 105a is configured to generate a first polarized multi-passband wavelengths DA, and the second conformal filter 105b is configured to generate a second polarized multi-passband wavelengths Σλ₂. In the exemplary embodiment, the first and second multi-passband wavelengths Σλ₁ and Σλ₂ are directed to a detector 115 through a lens assembly (not shown). In another embodiment, the first and second multi-passband wavelengths Σλ₁ and Σλ₂ may be combined as they are directed to the detector 115. In some embodiments, beam paths from the polarizing beam splitter 107 to the beam combiner 111 may be made symmetrical to avoid, for example, a need for infinitely-corrected optics.

In the exemplary embodiment, the detector 115 comprises a charge coupled device (CCD) detector. However, the present disclosure contemplates that the detector 115 may comprise other suitable detectors including, for example, a complementary metal-oxide-semiconductor (CMOS) detector, an indium gallium arsenide (InGaAs) detector, a platinum silicide (PtSi) detector, an indium antimonide (InSb) detector, a mercury cadmium telluride (HgCdTe) detector, or combinations thereof. Still referring to FIG. 1, the first and second conformal filters 105a and 105b may be tuned in unison to the same multi-passband wavelengths (Σλ₁=Σλ₂) using a controller 117. In another embodiment, the controller 117 may be configured to independently tune each multi-passband wavelengths Σλ₁ and Σλ₂ to respectively process orthogonal components of the input. Therefore, by appropriate control, the first and second conformal filters 105a and 105b may be tuned to either the same multi-passband wavelengths or different multi-passband wavelengths (Σλ₁≠Σλ₂). The controller 117 may be programmable or software-implemented to allow a user to selectively tune each of the first and second conformal filters 105a, 105b as desired. In the embodiment of FIG. 1, a fast switching mechanism (not shown) may be provided to switch between the two views (or spectral images) corresponding to spectral data collected by the detector 117 from each of the first and second conformal filters 105a and 105b. Alternatively, two such spectral views or images may be combined or overlaid into a single image to increase contrast or intensity, or for the purpose of comparison. The exemplary embodiment in FIG. 1 comprises a single CCD detector 115 to capture the filtered signals received from the conformal filters 105a and 105b.

FIG. 1B illustrates an alternative embodiment having a patterned conformal filter. In this embodiment, the beam combiner 111 and the first mirror 109a may be removed and two detectors 115 may be used. The first conformal filter 105a is configured to filter and transmit first multi-passband wavelengths corresponding to a T1 state to a first detector 115a that detects the first multi-passband wavelengths and generates a first image data set (T1). In similar fashion, the second conformal filter 105b is configured to filter and transmit second multi-passband wavelengths corresponding to a T2 state to a second detector 115b that detects the second multi-passband wavelengths and generates a second image data set (T2).

U.S. Pat. No. 9,157,800 to Treado et al., filed Jan. 15, 2014, issued Oct. 13, 2015, assigned to ChemImage Technologies LLC and entitled SYSTEM AND METHOD FOR ASSESSING ANALYTES USING CONFORMAL FILTERS AND DUAL POLARIZATION discloses the use of conformal filters in a dual polarization configuration as discussed above. This reference is hereby incorporated by reference in its entirety.

FIG. 1A illustrates an end-on view of the distal end of the endoscope/probe 102. The distal end features a lens 119 for collecting interacted photons 101 and fiber ends 121 of the fiber optic bundle 103 which illuminate the oral cavity 100 to generate the interacted photons 101. The detector 115 detects the multi-passband wavelength from the first and second conformal filters 105a and 105b and is configured to generate one or more image data sets. The image data set may comprise a T1 image corresponding to the first multi-passband wavelengths Σλ₁ and a T2 image corresponding to the second multi-passband wavelengths Σλ₂. In one embodiment, the image data set comprises a Raman image data set. The one or more image data sets generated by the detector 115 may be further analyzed as set forth below.

System Having MOE Filter Arrangements

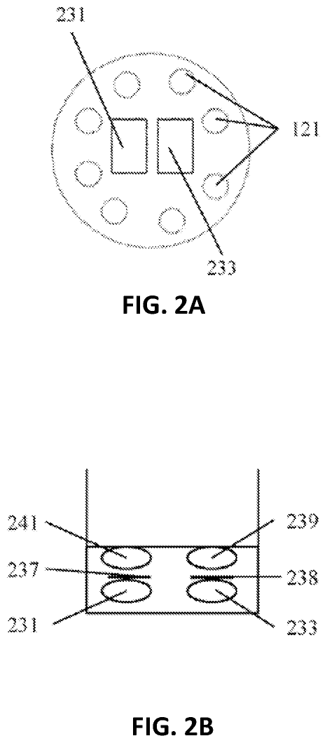

FIG. 2 illustrates another embodiment featuring modulating the collected optical signal. In FIG. 2, an illumination source 103 generates illuminating photons which traverse along a fiber optic bundle 104 through an endoscope/probe 102 and terminate at a series of fiber ends 121 on the distal end of the endoscope/probe 102 (shown in FIG. 2A). The fiber ends 121 emit illuminating photons to illuminate an oral cavity 100 to produce a plurality of interacted photons 101. The interacted photons are collected by a first collection optic 231 and a second collection optic 233. The first collection optic 231 collects a first portion of the interacted photons 101 and passes these photons on to a first Multivariate Optical Element (“MOE”) filter 237 which filters the first portion of the interacted photons 101 to generate a first portion of filtered photons. The first portion of filtered photons is detected by a first detector 241. Further, the second collection optic 233 collects a second portion of the interacted photons 101 and passes these photons on to a second MOE filter 238 to generate a second portion of filtered photons. The second portion of filtered photons is detected by a second detector 239. In one embodiment, the first detector 239 and the second detector 241 are CCD detectors. In other embodiments, the detectors 239 and 241 may comprise other suitable detectors including, for example, a complementary metal-oxide-semiconductor (CMOS) detector, an indium gallium arsenide (InGaAs) detector, a platinum silicide (PtSi) detector, an indium antimonide (InSb) detector, a mercury cadmium telluride (HgCdTe) detector, or combinations thereof.

In one embodiment, the first MOE filter 237 may be configured to generate a first filtered passband. In one embodiment, the first MOE filter 237 is configured to generate a first filtered passband consistent with a randomized target or background. In one embodiment, the second MOE filter 238 may be configured to generate a second filtered passband consistent with the target oral cavity 100. In embodiments where the first MOE filter 231 is configured to generate a first filtered passband corresponding to a randomized target or background, the second MOE filter 238 may be configured to generate a second filtered passband corresponding to the target oral cavity. This type of embodiment permits discrimination of both a target and a background.

MOEs are typically known in the art. An MOE features wide-band, optical interference filters encoded with an application-specific regression (or pattern) specific to a target. MOEs provide multivariate optical computing by performing the optical computation based on the pattern of the filter. In other words, MOEs are uniquely tuned to the pattern that needs to be measured using multivariate analysis on the filter as opposed to capturing multiple measurements at different wavelengths to estimate the full spectrum of a target and processing this information by applying multivariate statistics to the spectrum. Thus, MOEs increase throughput and efficiency over conventional filters, which can increase the speed of analysis. Suitable MOEs would be apparent to those of skill in the art in view of this disclosure.

The first detector 241 is configured to detect the first filtered passband from the first MOE filter 237 to generate a first image data set (T1), and the second detector 239 is configured to detect the second filtered passband from the second MOE filter 238 to generate a second image data set (T2). The first image data set and the second image data set may be further analyzed, as set forth below.

### Modulating Illumination Source Signal

The following embodiments feature systems and methods for modulating the illumination source signal prior to interaction with a sample.

System Having a Conformal Filter Arrangement

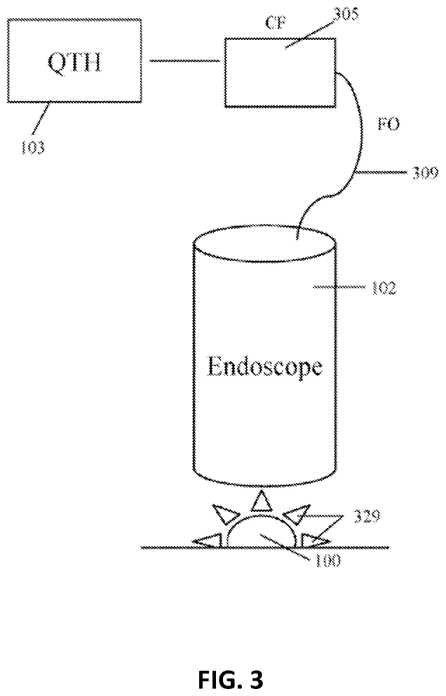

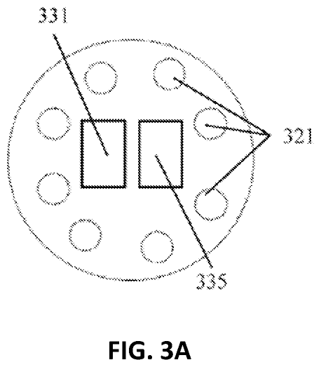

FIG. 3 illustrates an illumination source 103 configured to generate illuminating photons that are transmitted through a filter 305. In one embodiment, the filter 305 comprises a conformal filter. In some embodiments, the filter 305 may comprise other filters, such as a liquid crystal tunable filter (“LCTF”), a multi-conjugate filter, or other filters as would be apparent to those of skill in the art in view of this disclosure. The filter 305 may be controlled by a controller (not shown) that is configured to switch the filter configuration such that the filter initially passes first multi-passband wavelengths (Σλ₁) and subsequently passes second multi-passband wavelengths (Σλ₂). In one embodiment, the rate at which the controller switches between the two states is on an order of magnitude of a millisecond. The filter 305 transmits each of the first and second multi-passband wavelengths, Σλ₁ and Σλ₂, through a fiber optic bundle 309 to the distal end of an endoscope/probe 102 where each of the first and second multi-passband wavelengths exits the distal end of the endoscope/probe via fiber ends 321, as shown in FIG. 3A, to illuminate the sample 100 and produce interacted photons 329. The interacted photons 329 are collected by a first detector 331 and a second detector 335 located on the distal end of the endoscope/probe 102. The first and second detectors 331 and 335 may comprise CCD detectors. However, other detectors, such as those disclosed herein in reference to FIGS. 1-2, may be employed. The first detector 331 may be configured to detect substantially only the first multi-passband wavelengths. In one embodiment, the first detector 331 may be timed, i.e., turned off and on, to detect the first multi-passband wavelengths concurrent with the filter 305 transmitting the first multi-passband wavelengths. Likewise, the second detector 335 may be configured to detect substantially only the second multi-passband wavelengths. In one embodiment, the second detector 335 may be timed, i.e., turned off and on, to detect the second multi-passband wavelengths concurrent with the filter 305 transmitting the second multi-passband wavelengths. In another embodiment, the timing sequence of the modulation between the first multi-passband wavelengths and the second multi-passband wavelengths and the detection of the first multi-passband wavelengths and the second multi-passband wavelengths with the corresponding detector may be controlled by the controller (not shown). The first detector 231 detects the first multi-passband wavelengths and generates a first image data set (T1), and the second detector detects the second multi-passband wavelengths and generates a second image data set (T2). In one embodiment, the first image data set and the second image data set may be further analyzed as set forth below.

System Having a Conformal Filters in Dual Polarization Arrangement

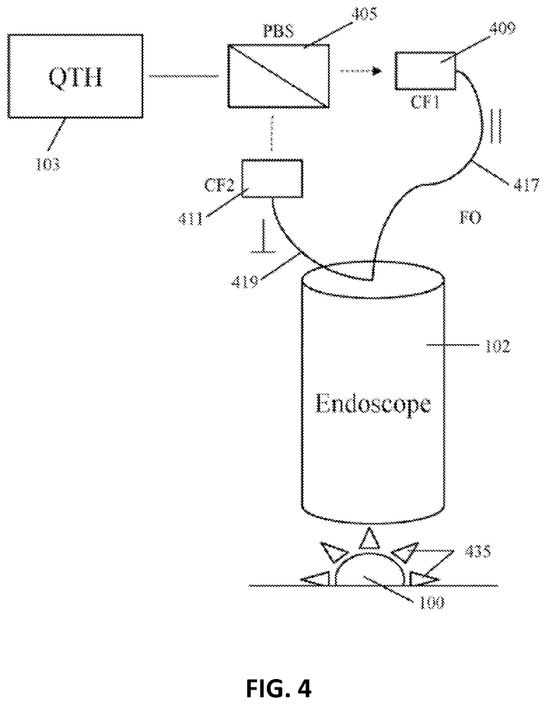

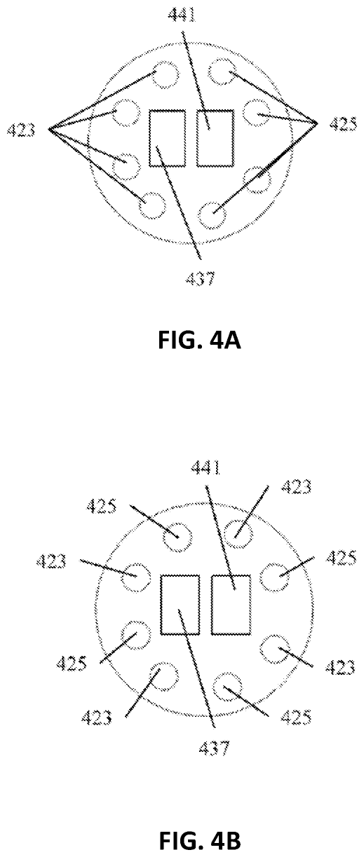

FIG. 4 depicts another illustrative system incorporating illumination source modulation. In this embodiment, an illumination source 103 generates an optical signal that is transmitted through a polarizing beam splitter 405 which splits the optical signal into a first polarization signal and a second polarization signal. The first polarization signal is transmitted to a first filter 409, and the second polarization signal is transmitted to a second filter 411. In one embodiment, the first filter 409 and the second filter 411 may each comprise a conformal filter, as described herein. In another embodiment, the first filter 409 and the second filter 411 may each comprise an LCTF. In one embodiment, the first filter 409 and the second filter 411 may each comprise a multi-conjugate filter. The first filter 409 is configured to filter the first polarization signal and transmit a first multi-passband wavelengths (Σλ₁), and the second filter 411 is configured to filter the second polarization signal and transmit second multi-passband wavelengths (Σλ₂). The first multi-passband wavelengths and the second multi-passband wavelengths are transmitted from their respective filters 409, 411 to the distal end of an endoscope/probe 102 via a first fiber optic bundle 417 and second fiber optic bundle 419. In one embodiment, the first fiber optic bundle 417 and the second fiber optic bundle 419 comprise a polarization-maintaining fiber optic bundle.

FIG. 4A and FIG. 4B illustrate different embodiments of the distal end of the endoscope/probe 102. The first fiber bundle 417 and the second fiber bundle 419 traverse through the endoscope/probe 102 to the distal end. The first fiber bundle 417 terminates at first fiber ends 423, and the second fiber bundle 417 terminates at second fiber ends 425. FIG. 4A illustrates one exemplary arrangement for the first fiber ends 423 with respect to the second fiber ends 425. In this embodiment, the first fiber ends 423 are distributed together on one side of the distal end of the endoscope/probe 102, and the second fiber ends 425 are distributed together on the opposing side of the distal end of the endoscope/probe. In an alternative embodiment depicted in FIG. 4B, the first fiber ends 423 and the second fiber ends 425 alternate around the distal end of the endoscope/probe 102. Additional suitable arrangements of the fiber ends would be apparent to those of skill in the art based on the teachings of this disclosure. The oral cavity 100 of a patient is illuminated by the first multi-passband wavelengths and the second multi-passband wavelengths emitting from the first fiber ends 423 and the second fiber ends 425, respectively, to generate interacted photons 435. The interacted photons 435 are detected by a first detector 437 and a second detector 441 disposed on the distal end of the endoscope/probe 102. In the illustrated embodiment, the first detector 437 and the second detector 441 are each CCD detectors. However, other suitable detectors, such as those disclosed herein, may be employed. Such detectors would be apparent to one of skill in the art in view of this disclosure. In one embodiment, the first fiber bundle 417 and the second fiber bundle 419 each comprise polarization-maintaining fiber bundles. In such an embodiment, polarizers (not shown) may be disposed adjacent to the first and second detectors 437, 441 and configured to differentiate between a T1 state and a T2 state on the basis of polarization. In some embodiments, the first and second detectors 437, 441 may be arranged for stereovision. In some embodiments, the first detector 437 is configured to detect substantially only interacted photons generated from the first multi-passband wavelengths, and the second detector 441 is configured to detect substantially only interacted photons generated from the second multi-passband wavelengths. As such, the location of the first fiber ends 423 and second fiber ends 425 with respect to the first detector 437 and the second detector 441 can be arranged to optimize the detection of the interacted photons corresponding to the first multi-passband wavelengths by the first detector 437 and the interacted photons corresponding to second multi-passband wavelengths by the second detector 441. Once the first detector 437 and the second detector 441 detect the interacted photons 435, the first detector 437 is configured to generate a first image data set (T1), and the second detector 441 is configured to generate a second image data set (T2). In one embodiment, the first image data set and the second image data set may be further analyzed.

### System Having an Acousto-Optic Filter Arrangement

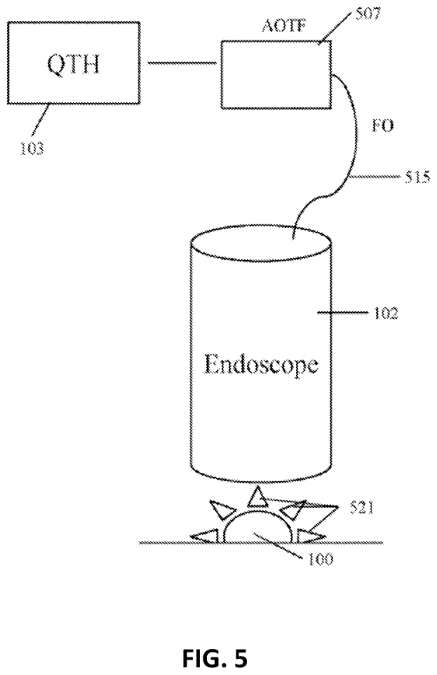

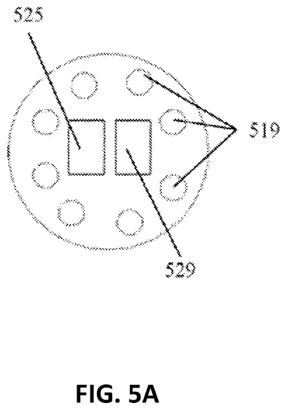

FIG. 5 illustrates an embodiment of the instant disclosure employing an acousto-optic tunable filter (AOTF). As depicted in FIG. 5, an illumination source 103 may be used to generate illuminating photons for illuminating an oral cavity 100 of a patient. A filter 507 is configured to filter photons emitted from the illumination source 103. In one embodiment, the filter 507 comprises an AOTF configured to transmit a single passband wavelength. The AOTF may be rapidly switched between target and background passband wavelengths in order to achieve a sampling rate of at least 10 frames per second. In another embodiment, the filter comprises a conformal filter based on AOTF technology in which the conformal AOTF transmits multi-passband wavelengths simultaneously. The conformal AOTF may be switched in series with microsecond switching speeds to switch between T1 and T2 states. In other embodiments, multiple conformal AOTFs may be employed in which the T1 and T2 states are selected simultaneously. In embodiments employing multiple acousto-optic tunable filters, each filter may be tuned to one or more wavelengths such that each filter transmits different multi-passband wavelengths simultaneously.

Acousto-optic tunable filters are known in the art and, generally, operate by passing a beam of source light through a substrate, typically quartz. The substrate is vibrated by a piezoelectric transducer modulator. An RF frequency is applied to the modulator, causing the substrate to vibrate. Source light or radiation is passed through the vibrating substrate, which causes the source light passing through the substrate to diffract. Such diffraction creates a filter gradient for the source light. The source light emitted from the acousto-optic filter can be filtered to a desired passband wavelength by the RF frequency applied to the piezoelectric transducer. Details on the operation of an acousto-optic tunable filter are described in more detail in Turner, John F. and Treado, Patrick J. “Near-Infrared Acousto-Optic Tunable Filter Hadamard Transform Spectroscopy” Applied Spectroscopy, 50.2 (1996), 277-284, which is hereby incorporated by reference in its entirety.

The passband wavelength transmitted from the filter 507 is transmitted to the distal end of an endoscope/probe 102 through a fiber optic bundle 515. FIG. 5A illustrates the distal end of the endoscope/probe 102 and features a plurality of fiber ends 519 from the fiber optic bundle 515. The fiber ends 519 transmit the passband wavelength from the filter 507 to illuminate an oral cavity 100 of a patient to produce interacted photons 521. The interacted photons 521 may be detected by a first detector 525 and a second detector 529 located on the distal end of the endoscope/probe 102. In some embodiments, only one detector may be used, i.e., the first detector 525, to detect a plurality of the interacted photons 521. In another embodiment, the interacted photons 521 are detected by both the first and second detectors 525, 529. In another embodiment, a plurality of acousto-optic tunable filters are employed. In such an embodiment, a first passband wavelength and a second passband wavelength may be generated. The first detector 525 may be configured to detect the first passband wavelength and generate a first image data set (T1), and the second detector 529 may be configured to detect the second passband wavelength and generate a second image data set (T2). In one embodiment, the first image data set and the second image data set may be further analyzed as set forth below.

### System Having an MOE Filter Wheel Arrangement

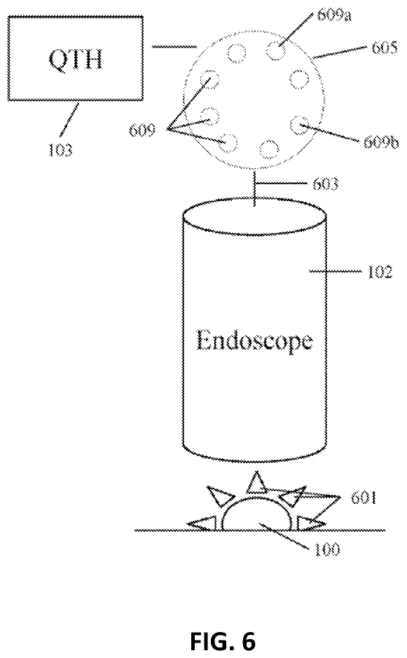

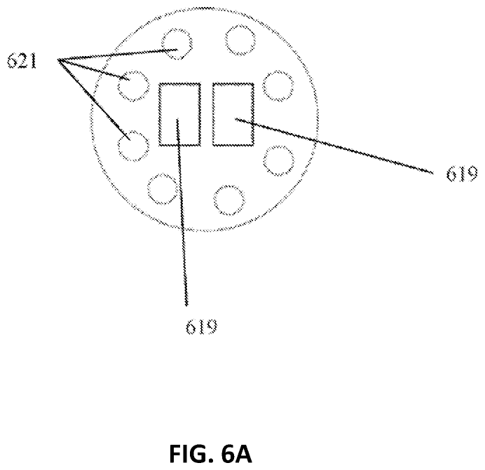

FIG. 6 illustrates another embodiment according to the instant disclosure. An illumination source 103 generates illuminating photons that are transmitted to a filter wheel 605 where the illuminating photons are filtered to generate filtered photons. The filter wheel 605 comprises a plurality of filter elements 609. In one embodiment, each filter element 609 comprises an MOE. Suitable MOEs for use according to the teachings described herein will be apparent to those of ordinary skill in the art. In some embodiments, each filter element 609 may be different from other filter elements and configured to filter and transmit a different passband wavelength. For example, a first filter element 609a may be configured to transmit a wavelength corresponding to a background, such as a specific type of tissue or anatomical structure, and a second filter element 609b may be configured to transmit a passband wavelength corresponding to an anomaly in a tissue sample, such as a cancerous tumor on the tissue. In this type of embodiment, the filter wheel 605 can be rotated during a surgical procedure to assist a surgeon in distinguishing normal tissue from cancerous tissue. In another embodiment, the filter elements 609 may be configured to detect a plurality of different samples. In one embodiment, the filter elements 609 may be configured to discriminate background tissue from cancerous tissue in an oral cavity.

The filtered photons are transmitted via a fiber optic bundle 603 to the distal end of the endoscope/probe 102 and exit the distal end of the endoscope/probe through a plurality of fiber ends 621 as shown in FIG. 6A. The filtered photons illuminate the oral cavity 100 of a patient and generate a plurality of interacted photons 601. The interacted photons 601 are detected by a one or more detectors 619, and the one or more detectors 619 are configured to generate an image data set (T1). In one embodiment, the image data set may be further analyzed, as set forth below.

### System Having a Patterned Etalon Filter Arrangement

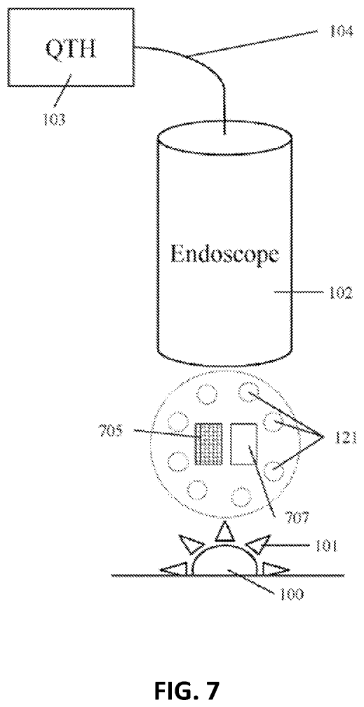

FIG. 7 depicts an illustrative system having a patterned etalon filter arrangement in accordance with an embodiment. In an embodiment, an illumination source 103 generates illuminating photons that are transmitted through a fiber optic bundle 104 to the distal end of the endoscope/probe 102 to fiber ends 121. The illuminating photons exit the fiber ends 121, illuminate the oral cavity 100 of a patient, and generate interacted photons 101 from the oral cavity 100 of the patient. In some embodiments, the interacted photons 101 are detected by a first detector 705 and a second detector 707 disposed on the distal end of the endoscope/probe 102. In one embodiment, the first detector 705 and the second detector 707 comprise hyperspectral cameras. In one embodiment, the detectors 705 and 707 comprise a Fabry-Perot interferometric (patterned etalon) filter configuration disposed on each pixel of the detector. Suitable examples of patterned etalon filter arrangements and associated detectors are available from Ximea Corporation of Lakewood, Colorado. The filter on each pixel may be configured to transmit one or more passband wavelengths for each pixel. In one embodiment, the first detector 705 comprises a patterned etalon filter arrangement in a mosaic snapshot arrangement. A mosaic snapshot can be acquired over 1088×2048 pixels. In one embodiment, the mosaic snapshot comprises a 4×4 mosaic having 16 wavelength bands. In another embodiment, the mosaic snapshot comprises a snapshot of the sample from 465 nm to 630 nm at 11 nm intervals. In another embodiment, the mosaic snapshot may comprise a 5×5 mosaic having 25 bands over a wavelength range from about 600 nm to 1,000 nm. In another embodiment, the mosaic snapshot may include a spatial resolution per band of about 512×272 with up to 2 megapixels with interpolation and may collect up to 170 data-cubes/sec.

In another embodiment, the first detector 705 and the second detector 707 may comprise a patterned etalon filter arrangement for obtaining a snapshot tiled configuration. In one embodiment, the snapshot tiled configuration transmits a passband wavelength at each pixel. The patterned etalon snapshot tiled filter configuration can acquire up to 1088×2048 pixels. In one embodiment, the tiled snapshot has a spectral resolution of up to 32 bands and can detect wavelengths ranging from 600 nm to 1,000 nm over 12 incremental steps. In another embodiment, the spatial resolution per band is about 256×256. In another embodiment, the tiled snapshot may detect up to 170 data-cubes/sec. The patterned etalon filter arrangement may also be customized to generate a predetermined response for an oral cavity and the desired result. Such customization would be apparent to one of skill in the art in view of this disclosure.

In one embodiment, the first detector 705 and the second detector 707 comprise mosaic filter arrangements developed by IMEC of Leuven, Belgium. In such an embodiment, the patterned etalon mosaic filter arrangements of the first detector 705 and the second detector 707 are configured to transmit one or more different wavelength bands at each pixel. In another embodiment, the first detector 705 and the second detector 707 comprise patterned etalon tiled filter arrangements. In such an embodiment, the patterned etalon tiled filter arrangements of the first detector 705 and the second detector 707 are configured to detect a different wavelength band at each pixel. In another embodiment, only a first detector 705 having either a snapshot mosaic patterned etalon filter arrangement or a snapshot tiled patterned etalon filter arrangement is used.

The first and second detectors 705, 707 are configured to generate one or more image data sets for each passband wavelength transmitted from the filter arrangements. In one embodiment, the first and second detectors 705, 707 are configured to generate a first image data set (T1) and a second image data set (T2), respectively. In one embodiment, the image data sets may be further analyzed, as set forth below.

### Other System Embodiments

In yet another embodiment, an illumination source may be configured to generate illuminating photons at specific wavelengths. For example, the illumination source may comprise a plurality of LEDs where a first portion of the LEDs are configured to generate a first wavelength and a second portion of the LEDs are configured to generate a second wavelength for illuminating an oral cavity of a patient. In such an embodiment, a first detector may be configured to detect interacted photons from the first wavelength and generate a first image data set (T1), and a second detector may be configured to detect interacted photons from the second wavelength and generate a second image data set (T2). Other illumination sources or arrangements may be employed which are capable of producing illuminating photons at a plurality of wavelengths. In one embodiment, the illumination source comprises a modulating laser which is capable of generating multiple wavelengths.

The image data sets described herein may comprise a visible-near infrared (Vis-NIR) image data set and/or a short-wave infrared (SWIR) image data set. In other embodiments, image date sets may additionally or alternately include one or more of an ultraviolet (UV) image data set, fluorescence image data set, a visible (VIS) image data set, a Raman image data set, a near-infrared (NIR) image data set, a mid-infrared (MIR) image data set, and a long-wave infrared (LWIR) image data set. In another embodiment, an image data set may comprise a hyperspectral image data set. The image data sets of the instant disclosure may further be analyzed.

In one embodiment, the systems disclosed herein may include a fiber array spectral translator (FAST). Suitable FAST devices are disclosed in U.S. Pat. No. 8,098,373 to Nelson et al., filed Apr. 13, 2010, issued Jan. 17, 2012, entitled SPATIALLY AND SPECTRALLY PARALLELIZED FIBER ARRAY SPECTRAL TRANSLATOR SYSTEM AND METHOD OF USE, and assigned to ChemImage Technologies LLC, the disclosure of which is incorporated by reference in its entirety.

In one embodiment, the systems disclosed herein may comprise a processor and a non-transitory processor-readable storage medium in operable communication with the processor. The storage medium may contain one or more programming instructions that, when executed, cause the processor to analyze the image data sets. In one embodiment, the analysis may comprise applying an optical computation to the data set. In another embodiment, the optical computation may comprise one or more of T1, and (T1−T2)/(T1+T2). Other optical computations known in the art may be applied. In one embodiment, the analysis may comprise applying one or more chemometric techniques to the image data sets. The chemometric analysis may comprise one or more of a multivariate curve resolution analysis, a principle component analysis (PCA), a partial least squares discriminant analysis (PLSDA), a k-means clustering analysis, a band t entropy analysis, an adaptive subspace detector analysis, a cosine correlation analysis, a Euclidian distance analysis, a partial least squares regression analysis, a spectral mixture resolution analysis, a spectral angle mapper metric analysis, a spectral information divergence metric analysis, a Mahalanobis distance metric analysis, and spectral unmixing analysis. In some embodiments, the processor may be configured to control operation of the system. For example, in embodiments where a tunable filter is employed, the process may be configured to cause the controller to apply voltages to the tunable filter to obtain the desired passband transmission. Further, the processor may be configured to control timing of an illumination source and detectors so that the correct detector is in operation for the specific illumination. Other processor configurations are contemplated and would be apparent to one of skill in the art in view of this disclosure.

The systems according to the instant disclosure may further include a display. In some embodiments, the display may include one or more results from one or more of the detectors. In another embodiment, the display may include one or more results from the analysis of the processor. In one embodiment, the display may include one or more results from one or more of the detectors and one or more results from the analysis of the processor.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A system for detecting oral cancer in vivo, the system comprising:
an illumination source configured to generate illuminating photons;
an imaging device comprising:
a fiber optic bundle comprising a plurality of optical fibers configured to receive the illuminating photons from the illumination source and direct the illuminating photons to an oral cavity of a patient, and
a lens configured to collect interacted photons from the oral cavity of the patient;
a polarizing beam splitter configured to receive the interacted photons from the lens and split the interacted photons into at least a first plurality of interacted photons and a second plurality of interacted photons;
a first tunable conformal filter configured to receive the first plurality of interacted photons and to generate first polarized multi-passband wavelengths;
a second tunable conformal filter configured to receive the second plurality of interacted photons and to generate second polarized multi-passband wavelengths;
a beam combiner configured to receive and combine the first and second polarized multi-passband wavelengths;
a detector configured to receive the combined first and second polarized multi-passband wavelengths; and
a controller configured to tune the first and second tunable conformal filters such that the first polarized multi-passband wavelengths and the second polarized multi-passband wavelengths discriminate between cancerous tissue and non-cancerous tissue in the oral cavity.

2. The system of claim 1, wherein the illumination source comprises at least one of a quartz tungsten halogen light, a metal halide light, a light emitting diode (LED), a LED array, a pulsed LED, a pulsed LED array, a laser, a pulsed laser, or a broadband illumination source.

3. The system of claim 1, further comprising:
a first mirror configured to direct the first polarized multi-passband wavelengths from the first tunable conformal filter to the beam combiner; and
a second mirror configured to direct the second polarized multi-passband wavelengths from the second tunable conformal filter to the beam combiner.

4. The system of claim 1, wherein the detector comprises at least one of a charge coupled device (CCD) detector, a complementary metal-oxide-semiconductor (CMOS) detector, an indium gallium arsenide (InGaAs) detector, a platinum silicide (PtSi) detector, an indium antimonide (InSb) detector, or a mercury cadmium telluride (HgCdTe) detector.

5. The system of claim 1, wherein the first polarized multi-passband wavelengths correspond to a background and the second polarized multi-passband wavelengths correspond to the cancerous tissue.

6. The system of claim 1, wherein the imaging device comprises an endoscope or a handheld probe.

7. A method of detecting oral cancer in vivo, the method comprising:
illuminating an oral cavity of a patient with a plurality of illuminating photons;
receiving a plurality of interacted photons from the oral cavity of the patient;
filtering the plurality of interacted photons into first polarized multi-passband wavelengths and second polarized multi-passband wavelengths using first and second tunable conformal filters, respectively;
capturing, via a detector, the first and second polarized multi-passband wavelengths; and
automatically discriminating between cancerous tissue and non-cancerous tissue in an image resolved from the first and second polarized multi-passband wavelengths.

8. The method of claim 7, wherein illuminating the oral cavity comprises illuminating the oral cavity with the plurality of illuminating photons from at least one of a quartz tungsten halogen light, a metal halide light, a light emitting diode (LED), a LED array, a pulsed LED, a pulsed LED array, a laser, a pulsed laser, or a broadband illumination source.

9. The method of claim 7, further comprising:
directing, via a first mirror, the first polarized multi-passband wavelengths from the first tunable conformal filter to a beam combiner; and
directing, via a second mirror, the second polarized multi-passband wavelengths from the second tunable conformal filter to the beam combiner.

10. The method of claim 7, wherein the detector comprises at least one of a charge coupled device (CCD) detector, a complementary metal-oxide-semiconductor (CMOS) detector, an indium gallium arsenide (InGaAs) detector, a platinum silicide (PtSi) detector, an indium antimonide (InSb) detector, or a mercury cadmium telluride (HgCdTe) detector.

11. The method of claim 7, wherein the first polarized multi-passband wavelengths correspond to a background and the second polarized multi-passband wavelengths correspond to the cancerous tissue.

12. The method of claim 7, wherein the oral cavity of the patient is illuminated with the plurality of illuminating photons via an endoscope or a handheld probe.

13. An imaging system for detecting oral cancer in vivo, the imaging system for use with an illumination source and an imaging device, the illumination source configured to generate illuminating photons, and the imaging device configured to direct the illuminating photons to an oral cavity of a patient and collect interacted photons from the oral cavity of the patient, the system comprising:
a polarizing beam splitter configured to receive the interacted photons from the imaging device and split the interacted photons into at least a first plurality of interacted photons and a second plurality of interacted photons;
a first tunable conformal filter configured to receive the first plurality of interacted photons and to generate first polarized multi-passband wavelengths;
a second tunable conformal filter configured to receive the second plurality of interacted photons and to generate second polarized multi-passband wavelengths;
a beam combiner configured to receive and combine the first and second polarized multi-passband wavelengths;
a detector configured to receive the combined first and second polarized multi-passband wavelengths; and
a controller configured to tune the first and second tunable conformal filters such that the first polarized multi-passband wavelengths and the second polarized multi-passband wavelengths discriminate between cancerous tissue and non-cancerous tissue in the oral cavity.

14. The system of claim 13, wherein the illumination source comprises at least one of a quartz tungsten halogen light, a metal halide light, a light emitting diode (LED), a LED array, a pulsed LED, a pulsed LED array, a laser, a pulsed laser, or a broadband illumination source.

15. The system of claim 13, further comprising:
a first mirror configured to direct the first polarized multi-passband wavelengths from the first tunable conformal filter to the beam combiner coupled to the detector; and
a second mirror configured to direct the second polarized multi-passband wavelengths from the second tunable conformal filter to the beam combiner.

16. The system of claim 13, wherein the detector comprises at least one of a charge coupled device (CCD) detector, a complementary metal-oxide-semiconductor (CMOS) detector, an indium gallium arsenide (InGaAs) detector, a platinum silicide (PtSi) detector, an indium antimonide (InSb) detector, or a mercury cadmium telluride (HgCdTe) detector.

17. The system of claim 13, wherein the first polarized multi-passband wavelengths correspond to a background and the second polarized multi-passband wavelengths correspond to the cancerous tissue.

18. The system of claim 13, wherein the imaging device comprises an endoscope or a handheld probe.
